(19) United States
(12) Patent Application Publication
(10) Pub. No.: US 2007/0128708 A1
(43) Pub. Date: Jun. 7, 2007

(54) Variable volume electroporation chamber and methods therefore

(75) Inventor: Andre S. Gamelin, Vista, CA (US)

Correspondence Address: BIOTECHNOLOGY LAW GROUP;C/O PORTFOLIOIP, PO BOX 52050, MINNEAPOLIS, MN, 55402, US

(73) Assignee: Genetronics, Inc., San Diego, CA (US)

(21) Appl. No.: 11/636,167

(22) Filed: Dec. 7, 2006

Related U.S. Application Data
(60) Provisional application No. 60/748,644, filed on Dec. 7, 2005.

(51) Int. Cl. C12N 13/00; C12M 1/42

(52) U.S. Cl. 435/173.6; 435/285.2

(57) ABSTRACT

Disclosed is a chamber apparatus for electroporating in vitro relatively large volumes of a fluid medium carrying biological-cells-or-vesicles wherein-a reservoir for carrying said cells and vesicles is variable in its volume on demand and wherein the volume chosen is directly related to the volume of the sample to be electroporated. The apparatus has further embodiments wherein the chamber is disposable and can be operated either in isolation from a patient or connected thereto.

### FIELD OF THE INVENTION

This invention relates to electroporation of cells and vesicles in vitro. More specifically, this invention relates to electroporation of cells and vesicles in an electroporation chamber, particular a disposable chamber having an “on-demand” variability in total volume.

### BACKGROUND OF THE INVENTION

The following description includes information that may be useful in understanding the present invention. It is not an admission that any such information is prior art, or relevant, to the presently claimed inventions, or that any publication specifically or implicitly referenced is prior art.

The electroporation arts are replete with ex vivo means of transfecting biological cells and vesicles. For example, U.S. Pat. No. 5,720,921 to Meserol discloses an electroporation chamber that is designed as a continuous flow chamber wherein vesicles are transferred to the chamber, electroporated and flushed out after electroporation pulses are applied. Other flow chambers include U.S. patents to Nicolau (U.S. Pat. No. 5,612,207), Dzekunov (U.S.P.A.N.2001/0001064), and Vernhes (U.S. Pat. No. 6,623,964). In the case of each of these disclosures, the flow chamber is not an optimal design for clinical applications of electroporating biological cells. This is because of mechanical problems that must be addressed for sterility and because it is difficult to correlate the electroporation of cell populations with the pulses that are used as cells continuously pass through the chamber.

Other electroporation chambers have also been disclosed wherein continuous flow of the medium carrying the vesicles is not used but the electroporation chamber device includes various elements. For example, U.S. Pat. No. 4,906,576 to Marshall discloses a chamber having among other elements a magnetic core. U.S. Pat. No. 6,897,069 to Jarvis discloses an electroporation sample chamber with removable electrodes. Other chambers are cuvette style for handling small samples, i.e., about 250 μl to 1.5 ml. Still other chambers, such as disclosed in WO04/083,379 to Walters, provide for larger volume, i.e. up to 10 or so milliliters but such a chamber is not dynamic in that the size of the chamber is fixed and the electrodes employed are two parallel plates that provide for use with only a fixed range of conductivity of the medium containing the vesicles which must be calculated relative to the volume/cell density and conductivity delivered into the fixed volume chamber. In the Walters patent, for example, the conductivity of the media is lowered so that large volumes can be processed and electroporated in a single electroporation event. Specifically, the medium is adjusted such that the medium has a conductivity in a range spanning 0.01 to 1.0 milliSiemens (resistance of 100-1000 Ohms).

In many cases it is undesirable or impracticable to adjust the conductivity of the medium as the most desirable mediums are saline based which are inherently conductive and provide a stable and viable environment for the cells. Saline based mediums are preferred since they are designed to provide an environment that closely resembles the natural habitat of the cells, thereby minimizing cell death. One of the most widely used media is Phosphate Buffered Saline (PBS) which is inherently conductive due to the ionic content of the solution.

The need to transfect cells of a patient ex vivo typically arises in gene therapy settings wherein cells, such as progenitor and other so called “stem” cells are removed from the patient and expanded many fold in number in cell culture. Since expansion of cells is typically carried out in suspension culture, the volume in which such cells are expanded is hugely variable. Additionally, cell density is important in the context of their survival in culture and with respect to the electroporation process. Thus, where it is desired to transfect large numbers of cells, volume differentials between individual samples can be highly variable. This means that in the clinical setting a mechanism must be used to add processing steps to make uniform the volumes between samples so that an electroporation chamber of a fixed size can be used, or there must be a mechanism for accommodating the variance in such medium volume without a direct need to adjust either the volume or cell density and/or to accommodate specific low conductivities of the medium.

Additionally, cells in suspension have traditionally been electroporated using a media having a relatively high conductivity, such as for example, phosphate buffered saline (PBS), which has a conductivity of 0.017 Siemens/cm. When attempting to electroporate large volumes of media containing viable biologic cells, even considering use of a chamber having a 0.4 cm gap, such a large current would be required to pulse the entire volume at one time due to the low resistance arising from attempting to pulse through a large cross-sectional area, the cells would likely be damaged, or be subjected to variability in the pulse conditions. Whereas some inventions have attempted to overcome such difficulties by designing systems that use low conductivity medium, the use of such a system is impractical as the low ionic strength media may harm cell viability. As noted recently in Current Gene Therapy (Vol. 5, pp 375-385, 2005) current electroporation methodology is not a feasible tool to transfect certain cells because of poor cell survival (approximately only 1%) unless a more cell friendly medium was employed. However, even prior improved medium conditions only provided for survival of 5-6% of the cells.

Consequently, there is a need in the art to advance the use of an ex vivo system for transfecting cells and vesicles such as stem cells with molecules in a large volume format, i.e., for example about 1 to up to 100 ml that can easily and inexpensively deliver to the cells such molecules without having to make, in each instance, critical computations for ionic strength vs. volume vs. cell density. Thus, the current invention addresses such a need by providing a system that is dynamic in its capacity for electroporating cells at any such volume.

### SUMMARY OF THE INVENTION

In a first embodiment the present invention provides an apparatus for electroporating cells and vesicles, particularly, antigen presenting cells, progenitor cells and/or stem cells ex vivo in large volume. Generally, such volume is between 1 and 100 ml, typically between 5 and 75 ml and preferably between 10 and 50 ml. By stem cells is meant pluripotent cells derived from either an embryo or adult sources that maintain a phenotype that can be induced to differentiate into various cell types including endoderm, mesoderm and ectoderm (Mendez et al., 2005). Other useful applications include the transfection of cells of the immune system for vaccination and therapeutic purposes. Cells of the immune system that may be transfected with the present invention include monocytes, macrophages, T and B lymphocytes, dendritic cells and other antigen presenting cells. While the present invention is directed at use of human cells, cells of other species can be processed with the present invention.

In a second embodiment the present invention provides an apparatus for electroporating cells and vesicles wherein the apparatus comprises a chamber that has an on-demand capability to assume any incremental volume between 1 and 100 ml. In a related embodiment, the apparatus may be operated at any such volume without needing to adjust or calculate for specific ionic strength relative to the volume or surface area of electrodes in contact with the medium carrying said cells or vesicles.

In a third embodiment the present invention chamber comprises a multiplicity of individually addressable electrodes, which in a preferred embodiment, allow for the capability of initiating electric pulses to the volume of fluid medium without having to calculate electrode gap to volume ratio as would likely be necessary if only a single electrode pair which spanned the entire chamber were used. Specifically, for any volume used, pulsing conditions (i.e., voltage, pulse shape, and duration of pulse) are independent of said volume. In a related embodiment, the conductivity of the fluid medium containing the cells may comprise any level of conductivity useful in the practice of electroporation of biological cells and vesicles. For example, the conductivity of the cell containing medium can be equivalent to phosphate buffered saline (PBS) or less.

In another embodiment, the invention electroporation chamber can accommodate fluid volumes without exposure to an open air environment and therefore can be operated without concern or need for an air filter or air bleed orifice designed into the chamber.

In another embodiment, the multiplicity of electrodes comprise a series of parallel “plate” electrodes that can be arranged within the invention chamber such that the lengths of said plates run in either the same direction as the corresponding variable volume adjustability, i.e., the direction of the push and pull of the plunger, or can run in a direction 90 degrees to the direction in which the volume of the chamber is expanded. In a related embodiment, the individual electrode plates can comprise any useful biocompatible and conductive material including titanium and gold. In a further preferred embodiment, the plates can comprise a width dimension that is generally greater than the distance, or gap, between opposing electrodes, and even more preferably greater than twice the gap distance. Each electrode plate can be individually addressable with an electric pulse sufficient to electroporate biological cells and vesicles lying in solution between any of the cathode and anode electrode plate pairs. In another embodiment, the electrodes can comprise an array of between 2 and 100 cathodes and 2 and 100 anodes, there always being an even number of cathodes and anodes so as to form pairs of positive and negative electrodes.

In still a further embodiment, the cathode and anode electrodes can be space on opposing interior sides of the reservoir at a distance of between 0.4 and 1 cm apart, i.e., the gap across which the electric pulse must transmit is about between 0.4 cm and 1 cm.

In still further embodiments, each pair of said anodes and cathodes can be energized at a load resistance (in Ohms) of between 2.4 and 29.5 Ohms depending upon the chamber size. When each electrode pair in the chamber is sequentially energized, the biological cells suspended in the chamber, regardless of their location in the chamber, will be pulsed with equivalent energy sufficient for electroporation to occur without damaging the cells.

In a further embodiment, the invention can include a variety of instrumentation or other features such as an indicator for detecting and displaying notice of completion of an electroporation pulse sequence imparted to the series of electrodes exposed to cell medium. Such an indicator is valuable for the user to keep track of whether a chamber had been exposed to a pulse. In another example, the chamber can include in its design a keying feature to assist the chamber in being seated into its base tray in a proper orientation so that as pulses are imparted onto each electrode in the proper sequence.

Other features and advantages of the invention will be apparent from the following drawings, detailed description, and appended claims.

### BRIEF DESCRIPTION OF THE DRAWINGS

FIG. 1 is perspective drawing showing the variable volume invention chamber 10 and an electrode energizing tray 200. As depicted, the chamber 10 is constructed to removably attach or mount onto the tray 200 such that the electrode contact nub 15, of each electrode plate 11 of the chamber contacts tray electrode tabs 201. In this embodiment, the contact nubs 15 are shown exiting from the chamber housing from the bottom or floor side of the chamber.

FIG. 2 is a drawing of the invention chamber depicting an end view of the chamber on the side of port 14 which port can be constructed in any manner to accommodate connection to a source of fluid medium containing cells to be electroporated such as, for example, a luer fitting.

FIG. 3 is a perspective drawing showing an exploded view of the invention chamber 10 wherein is shown plunger 12 with push rod 13 and semi-resilient cushion 16 which collectively slidably engage the internal walls (sides, top and floor) of the chamber 10 thereby providing a seal so as to allow fluid to be drawn into and pushed out of the chamber similar to a syringe. Electrodes 11 line opposing sides of the chamber 10. The drawing further shows electrode contact nubs 15, in this embodiment, projecting from the side of the chamber housing.

FIG. 4 is a drawing showing a partial perspective view of the end of chamber 10 comprising port 14. The plunger with its semi-resilient cushion can be positioned to create various volumes within the chamber.

FIG. 5 is top view of the invention chamber 10 showing the plunger 12 has been positioned about half volume 17 of the chamber

FIGS. 6A-E show a top view as in FIG. 5 and depict a step-wise pulsing of electrode pairs 2 to 6 (FIGS. 6A-E) such that the electric field 18 between each electrode pair is relatively uniform across the gap distance between the electrodes. Asterisks indicate the electrode pairs being energized.

FIG. 7 is a perspective drawing of an alternate chamber design wherein invention chamber 100 is constructed with relatively small surface area electrodes 111. Such a construct can be used in chamber constructs with a gap distance between the electrodes of about 1 cm.

FIG. 8 shows the mean fluorescence readings from cells treated as described in the example.

### DETAILED DESCRIPTION OF THE PREFERRED EMBODIMENTS

As those in the art will appreciate, the following description describes certain preferred embodiments of the invention in detail, and is thus only representative and does not depict the actual scope of the invention. Before describing the present invention in detail, it is understood that the invention is not limited to the particular device arrangements, systems, and methodologies described, as these may vary. It is also to be understood that the terminology used herein is for the purpose of describing particular embodiments only, and is not intended to limit the scope of the invention defined by the appended claims.

This invention involves ex-vivo methods of electroporation of mammalian cells and other vesicles, particularly stem and progenitor cells wherein the cells are suspended in a conductive media within a large volume chamber. The large volume chamber comprises multiple electrode pairs arranged in a manner that allows for the media to be exposed to multiple sequential pulses of electrical energy between each successive pair of opposing electrodes in that portion of the chamber that is exposed to said fluid medium. In such a chamber, the full volume of the medium containing the biologic cells is not electroporated all at one time but instead is electroporated in portions by pulsing individual pairs or alternatively groups of pairs of electrodes. Such pulsing can be sequentially, in single or multiple pairs, or staggered pulsing of more than one pair, e.g., for example, pulsing a first and a second pair of electrodes followed by pulsing of the second and the third pair, followed in turn by pulsing the third and the fourth pair, etc.

In a preferred embodiment, the large volume chamber of the invention provides for dividing the electric pulse load from a single pulse for the entire chamber down to a series of smaller loads to avoid physical limitations that naturally occur due to maximum limits of energy that can be applied electrodes of a given surface area, especially where high conductivity media is used. In a related embodiment, the chamber invention also allows for avoiding special handling requirements that would otherwise be necessary if a multiplicity of individual single standard cuvettes were employed, or if a specially selected low ionic strength media were employed. Stepping down the pulse load can be accomplished in an electroporation of a fixed dimension but given the practical need to accommodate a variety of volumes, the present invention overcomes the need to adopt special handling requirements that would be necessary in a chamber of fixed size, such as for example, volume adjustments, changes in ionic strength due to volume adjustment, and pumps or other means necessary to transport medium into and out of such a chamber.

In another embodiment, the invention comprises a method of using the invention chamber wherein the conductivity of the cell carrying medium is greater than 50 milliSiemens (Resistance less than 20 ohms) and even greater than 500 milliSiemens (Resistance less than 2 Ohms). This in contrast to conventional systems such as produced by Bio-Rad (i.e., the Gene Pulser Xcell Electroporation System) which is specified to operate at greater than 20 ohms, i.e., the conductivity of the medium (a low conductivity) preferred for such a device is less than 50 milliSiemens. Although use of a high conductivity solution can result in arcing or be related to other performance issues, such problem would only likely occur if the electroporating pulse were delivered to the entire chamber at one time. However, the present invention is not susceptible of arcing due to the fact that it uses a series of pulses from individual electrode pairs thereby stepping down the electric pulse load on any given segment of the total volume being electroporated.

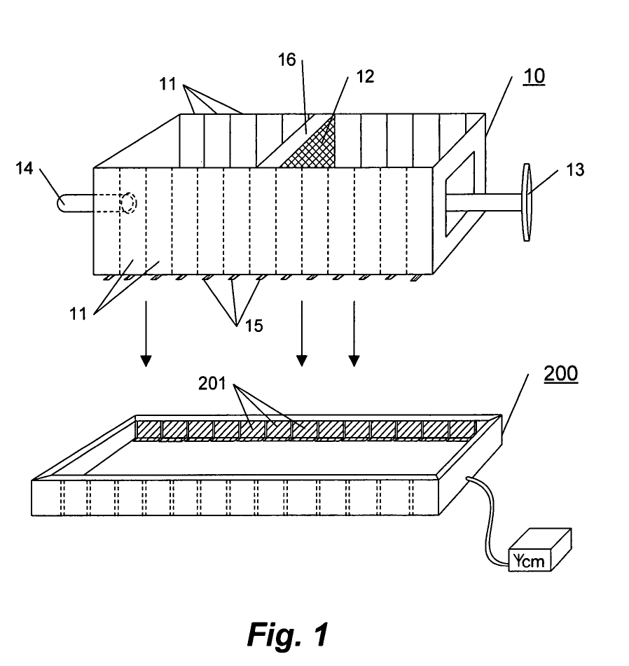

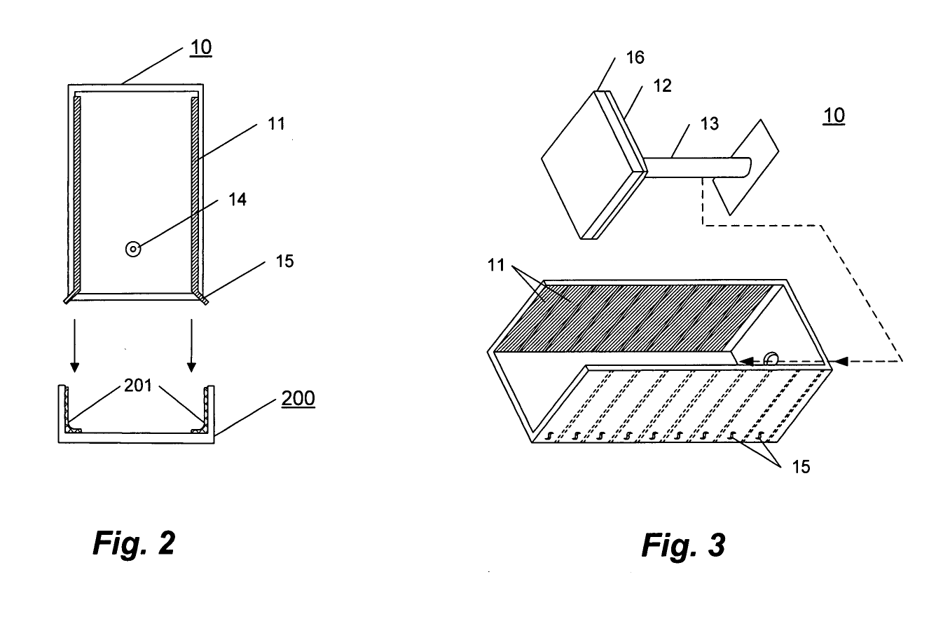

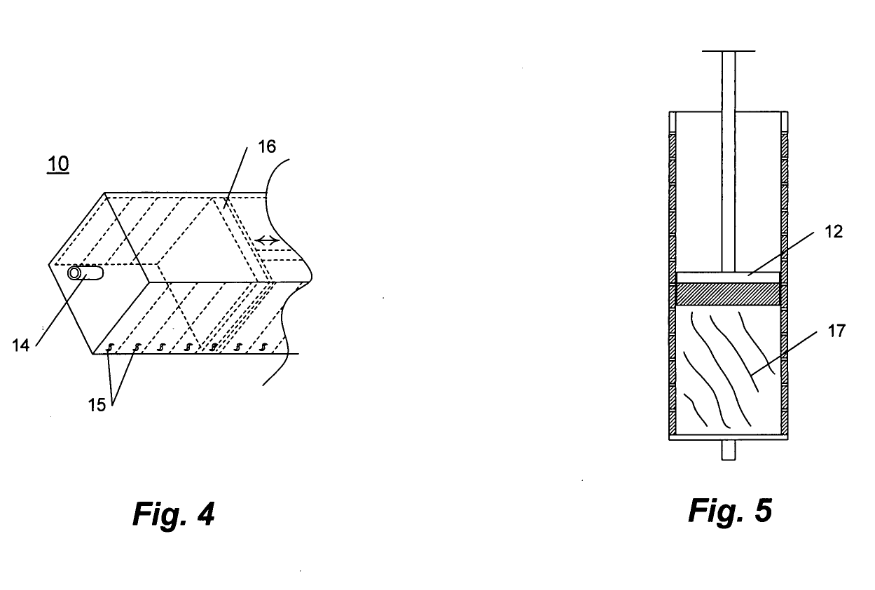

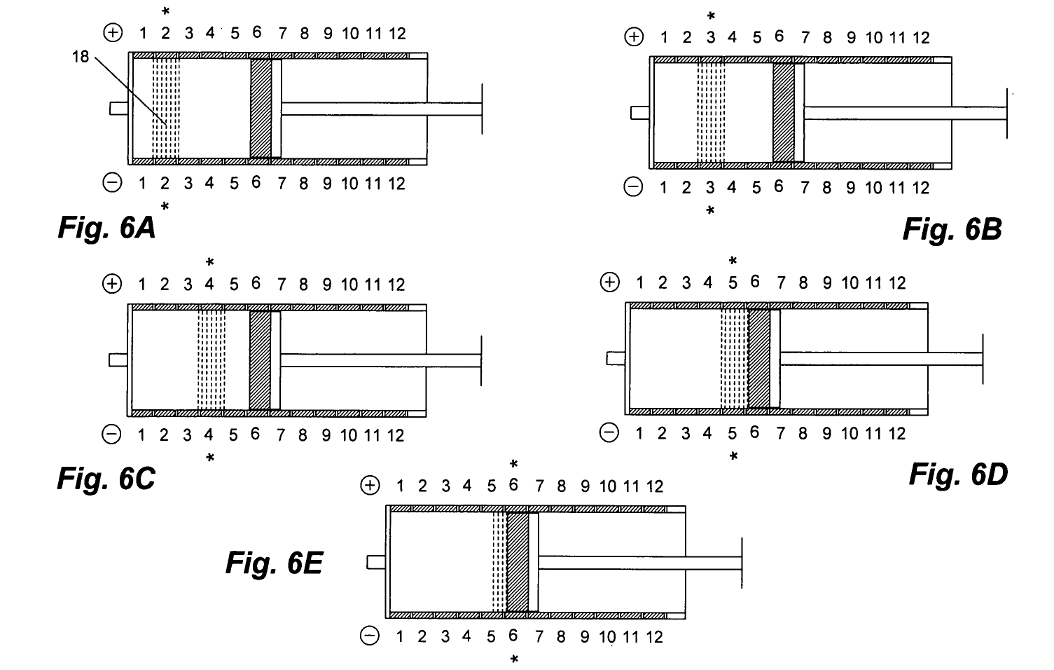

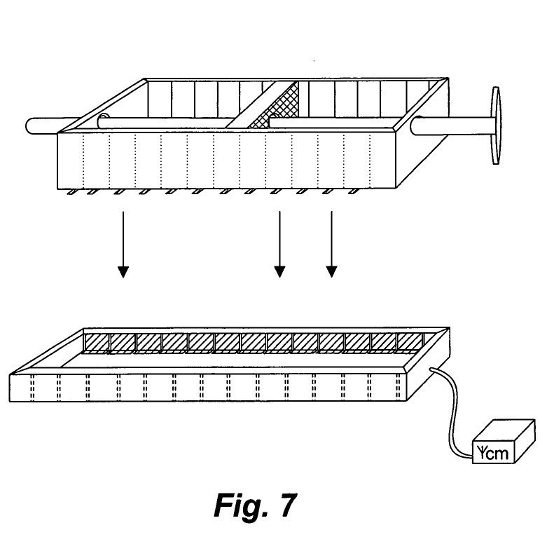

Turning now to the invention chamber, in a first preferred embodiment, as shown in FIG. 1, the chamber 10 comprises preferably a rectangular shaped chamber the interior volume of which, depending upon its construction, can have a capacity for accepting volumes of fluid medium up to and even greater than 100 ml. In practical terms, although the design of the large volume chamber allows for volume scale-up of any volume, typically the invention device is constructed to handle volumes normally experienced in the laboratory and clinical setting, i.e., volumes of less than 100 ml. Thus, typically, the invention chamber can be preferably constructed to hold maximum volumes of 5, 10, 15, 20, 25, 30, 35, 40, 50 or even 100 ml or any incremental volume of fluid medium between 5 and 100 ml.

The invention chamber 10 is constructed similar to a syringe and plunger wherein the rectangular chamber is increased or decreased in its volume capacity by inserting into said chamber a rectangular shaped plunger 12. The rectangular plunger is constructed in typical syringe plunger fashion wherein attached to the chamber side of the plunger is a semi-resilient inert rubber cushion 16 and on the other side is a plunger rod 13. Located near the end of the chamber opening where the plunger is inserted into the chamber interior, there is at least one plunger stop formed by either a tab or the end wall of the chamber itself, which keeps the plunger from being fully removed from the chamber interior. The chamber interior is accessible via port 14 which can be located in the end wall of the chamber or alternatively near the end wall but on the top, bottom or side walls.

The chamber further comprises a multiplicity of opposing anode and cathode electrodes 11. In a preferred embodiment, the distance or gap between opposing cathode and anode electrodes, i.e., the electrodes being on opposite sides of the chamber, is between 0.4 cm and 0.1 cm. In particularly preferred embodiment, the width dimension of each electrode is greater than the measurement of the gap between opposing electrodes and preferably greater than twice the gap distance. Thus, the width of the electrodes can be in the range of 0.4 to 5 cm. This feature provides for the intensity of the electric field to remain relatively uniform over the gap distance, whereas if the distance was greater than the width of the electrodes, the electric field would be subject of significant diminishment. In a related embodiment, the electrodes 11 can be arranged in the chamber either perpendicular to the pull of the plunger, or set in the chamber such that they extend the length of the chamber parallel to the direction of the plunger pull.

As further shown in FIG. 1, the chamber electrodes 11 are energized with electroporating pulses by setting the chamber into a base contactor tray 200 which provides for contact between the electrodes in the chamber and electrode contacts in the base tray 200 and source of electrical energy. The base contactor tray 200 can include additional embodiments for controlling such as the sequence of electrode pulsing. Alternatively, the controls for electrode pulsing can be integrated into the electrical pulse source, i.e., the electroporation generator.

In still another embodiment the invention chamber can be constructed with any number of electrode pairs (i.e., a pair comprising an anode and a cathode) but preferably the number of pairs will depend on the surface area of each electrode vs the gap between them. This is because of the physical limitation on the amount of electrical load that can be placed across a gap of a given dimension without arcing in the presence of a cell medium having a conductivity in the physiologic range, i.e., an ionic strength range similar to phosphate buffered saline (PBS). For example, as shown in the Table I, electrodes can be designed having various surface areas for use with various gap distances to electroporate samples using a variety of pulsing conditions. In each case, the actual volume electroporated is irrelevant to the actual pulsing conditions because the chamber is constructed to provide for use of electrodes at a pulse load easily within a range that is well below the maximum load that would be necessary if electrodes were all pulsed simultaneously. Thus, in a preferred embodiment, the electrodes can be constructed with surface area dimensions of between 0.8 and 20 cm². For another example, for chambers having a maximum volume of 20 ml the number of electrodes can be between 1 and 50 each having a surface area of between 1 and 20 cm² depending upon the gap distance between the opposing electrodes.

TABLE I

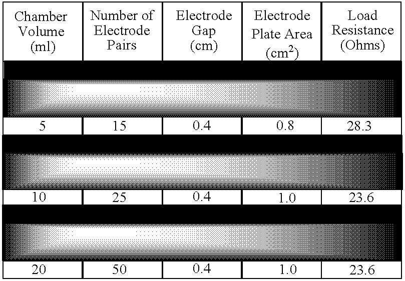

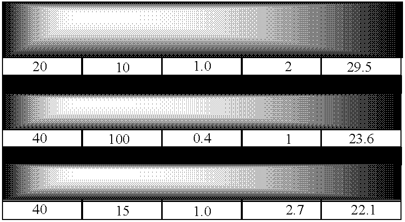

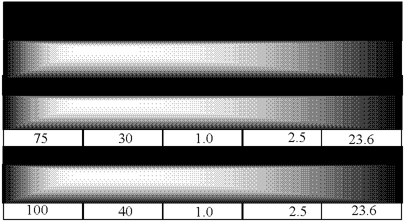

No shading = Desirable electroporation load; gray shading = Workable electroporation load; dark shading = Un-workable electroporation load

As indicated in Table I, chambers can be constructed with a variety of maximum volume capacities, a variety of electrode gaps, a variety of number of electrodes that provide for stepping down the electrical load per pulse, and at the same time remain compatible with cell media conductivity in the physiologic range.

One of the most widely used media for electroporation is PBS which is inherently conductive due to the ionic content of the solution. Since PBS has a conductivity of 0.017 Siemens/cm, use of PBS in a standard 0.8 ml cuvette would create a resistance load of approximately 12 ohms. Performing electroporation with load resistances less than 100 ohms is difficult to achieve as most conventional electroporation equipment can not operate in ranges of low resistance. For example, electroporation equipment by Biorad, specifically, the Gene Pulser Xcell, has a published lower load limit of 20 ohms. Other equipment such as BTX electroporation generators have limitations based on the inherent capabilities of the equipment, wires and connections. It is not unreasonable to expect 0.2 ohm to 1 ohm resistance in the wires, connectors and capacitors. When a 2 ohm load is pulsed in such a system as much as 33% of the electroporation voltage (and energy) could be lost in the equipment. Additionally, low resistance loads also present other complications due to the requirement for large surge currents. These complications could further include transient switching signal noise, instrument reliability and sample heating.

With respect to the current invention, we have found that dividing the load down to manageable levels with a load resistance of 2 ohms or greater allows for pulsing individual electrodes in sequence rather than pulsing a single electrode pair for the entire volume of medium to be electroporated. In a related embodiment, the use of physiologic ionic strength provides for a simplification of the electroporation process as cells can be extracted from cell culture, washed with PBS, and placed directly into the variable volume chamber.

In use, a patient cell population sample, such as an expanded population of stem cells or other progenitor cells is prepared for dispensing into the chamber as one of skill in the art would understand. Typically, the medium in which the cells are processed have an ionic strength equivalent to physiological saline. Additionally, depending upon the particular cell sample, the volume of the sample would likely be in a range of 5 to 50 ml.

Upon filling the chamber with the cell containing medium, the chamber is placed in the base tray and a sensor incorporated into the tray identifies the number of electrodes that are exposed to the fluid medium. In embodiments comprising detectors of load, or exposure of electrodes to the fluid medium, the detector can measure such elements as current. Then, having detected the appropriate electrodes to pulse, each opposed pair of electrodes exposed to the medium are then pulsed stepwise from one end of the chamber to the other. Alternatively, the electrodes can be pulsed in a variety of formats. For example, rather than pulsing one pair of opposing electrodes step-wise one after the other, the electrodes can be pulsed two opposing electrode pairs simultaneously followed by pulsing a second two opposing pairs. The electrodes can further be pulses in an overlap format wherein, for example after pulsing two opposing pairs of electrodes, the next electrode to be pulsed can be pulsed simultaneously with the adjacent electrode that had just been previously pulsed. In each case, the format of pulsing will likely provide sufficient electrical energy to electroporate all cells in the sample. Additionally, each of the manipulations of filling the chamber, movement of the plunger, and activation of the electrodes can all be accomplished by inanimate means, such as by electronics or motors as would be well understood by one of ordinary skill in the art.

### EXAMPLE

The following Example is provided to illustrate certain aspects, embodiments, and applications of the present invention, and to aid those of skill in the art in practicing it. This example does not in any way limit the scope of the invention in any manner.

This example describes a series of experiments using a series of three cuvettes versus a single cuvette.

Murine B16 cells (ATCC CRL 6475) were cultured as monolayers in standard tissue culture flasks in Mcoy=s media supplemented with 10% fetal bovine serum and 90 g/ml gentamicin. Cells were removed from the flasks using a solution of 0.05% trypsin and 0.02% EDTA. After removal, cells were washed three times in phosphate buffered saline (PBS) by cetrifugaton at 225×g and suspended in a small volume of PBS. The resulting suspension was enumerated using a standard hemacytometer and trypan blue exclusion dye. The cells were approximately 90% viable. The concentration of cells in the enumerated suspension was adjusted to 1×10⁶ cells/ml.

Cells were mixed in a 1:1 ratio with 120 μM freshly prepared calcein solution (in PBS) and subjected to electrical treatment in using a BTX T820 electroporation pulse generator. Cells were treated in standard 4 mm gap electroporation cuvette or a triple cuvette made by closely juxtaposing three 4 mm gap electroporation cuvettes, with a plexiglass spacer inserted between the center cuvette and each adjacent cuvette. Before assembly, the plexiglass spacers and sides of the sides of the center and end cuvettes to be juxtaposed were machined so that fluid could flow between the three cuvettes. Three different models of triple cuvettes were used. One had a 2 mm spacing between adjacent cuvettes, another had a 3 mm spacing between cuvettes, and the third had 4 mm spacers between adjacent cuvettes.

Pulses were applied to the standard 4 mm gap cuvette by applying one electrode as the anode and the other as the cathode that are integrated into the device. However, pulses were applied to the triple cuvettes in a very particular manner. Pulses were first applied across the 4 mm gap of an end cuvette. Pulses were next applied across the 4 mm gap of the center cuvette. Finally, pulses were applied across the 4 mm gap of the other end cuvette. A manual switch box was used to direct pulses from the BTX T820 electroporation power supply to the triple cuvette.

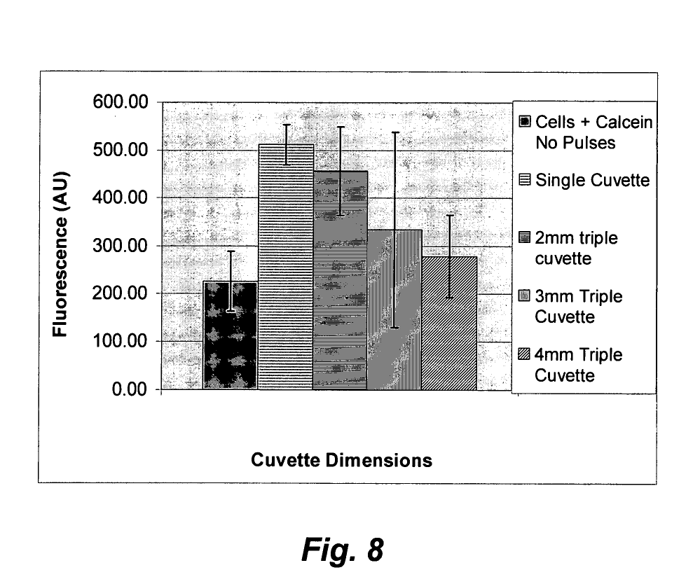

B16 cells mixed with calcein were treated in the single and all three triple cuvettes by applying eight direct current pulses with a nominal field strength of 1600 V/cm. For the single cuvette, one set of 8 pulses was applied. For each of the triple cuvettes, three sets of 8 pulses were applied. One set was applied across the 4 mm gap of each joined cuvette. After electrical treatment, the B16 cells were removed from the cuvettes and incubated at 37° C. for 20 minutes. The cells were washed three times in PBS, with pelleting between washes by centrifugation (225×g). After washing, the cells in each sample were resuspended in 400 μl of PBS and analyzed spectrafluorametrically using a fluorescence micro titer plate reader (Biotek). This analysis included analyzing three 100 μl aliquots of cell suspension from each sample. Mean fluorescence data was calculated to arrive at a single reading for each sample. Mean data from triplicate samples were pooled. FIG. 8 shows the mean fluorescence readings from cells exposed to calcein in (no pulses), cells exposed to calcein and pulsed in the single chamber, and cells exposed to calcein and pulsed in the three triple chambers. The data indicate that applying electric fields in all four types of cuvettes resulted in increased cellular fluosrecence relative to cells that were only exposed to calcein.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, the described embodiments are to be considered in all respects only as illustrative and not restrictive. All similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that use of such terms and expressions imply excluding any equivalents of the features shown and described in whole or in part thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A variable volume electroporation chamber for electroporating biological cells and vesicles in a suspension medium comprising:
A housing forming a reservoir wherein said housing comprises four side walls, a top and a bottom of said reservoir;
A valve comprising an external orifice and an internal lumen connecting said orifice to said reservoir, said connection of said lumen to said reservoir located in said housing;
A slidably adjustable means within said reservoir for aspirating into or expelling out of said reservoir a fluid medium through said lumen and orifice;
A multiplicity of spaced elongate cathode and anode electrodes placed parallel with respect to one another and along at least two opposing inner side walls of said reservoir wherein said electrodes are individually addressable with an electric pulse; and
A source of electrical energy connected to said electrodes, wherein said electrodes can be pulsed with sufficient electrical energy to cause electroporation of biological cells and vesicles contained within a fluid medium in said reservoir.

2. A variable volume chamber according to claim 1 wherein said slidably adjustable means comprises a plunger.

3. A variable volume chamber according to claim 1 wherein said chamber comprises a rectangular shape.

4. A variable volume chamber according to claim 1 wherein said chamber comprises a cylindrical shape

5. A variable volume chamber according to claim 3 wherein said plunger comprises a rectangular shape.

6. A variable volume chamber according to claim 4 wherein said plunger comprises a cylindrical shape.

7. A variable volume chamber according to claim 1 wherein said orifice comprises a stop cock and a connector for attaching to an external tube or other device.

8. A variable volume chamber according to claim 1 wherein said multiplicity of electrodes comprise anodes and cathodes, said anodes aligned along one inner side wall of said reservoir and said cathodes aligned along the inner side wall of an opposing side.

9. A variable volume chamber according to claim 8 wherein said electrodes are aligned parallel to the longitudinal direction along which the plunger can slidably travel in said reservoir.

10. A variable volume chamber according to claim 9 wherein said cathode and said anode electrodes are spaced on opposite sides of the reservoir between 0.4 and 1.0 cm apart.

11. A variable volume chamber according to claim 8 wherein said electrodes are aligned perpendicular to the longitudinal direction along which the plunger can slidably travel in said reservoir.

12. A variable volume chamber according to claim 11 wherein said cathode and said anode electrodes are spaced on opposite sides of the reservoir between 0.4 and 1.0 cm apart.

13. A variable volume chamber according to claim 8 wherein said multiplicity of electrodes comprise between 3 and 20 cathodes and between 3 and 20 anodes, the number of cathodes and anodes always being equal to one another.

14. A variable volume chamber according to claim 8 wherein said cathode and anode electrodes comprise plates having a width of between 0.4 and 5 cm.

15. A method of electroporating biological cells and vesicles in a liquid medium ex vivo to deliver to said cells and vesicles molecules of interest comprising:
Placing said cells/vesicles and said molecules of interest in an electroporation chamber of claim 1 wherein said cells/vesicles and molecules are aspirated into said reservoir by sliding the plunger in a direction to direct said liquid medium into said reservoir;
Setting the chamber in a mount comprising a source of electrical energy and activating said source of electrical energy to impart at least one electroporating pulse of electrical energy on to each pair of cathode and anode electrodes exposed to said liquid medium;
Expelling said cells/vesicles containing said molecules from said reservoir by sliding the plunger in a direction to force said cells/vesicles out of said reservoir thereby delivering to said cells and vesicles said molecules of interest.

16. The method of claim 15 further comprising a detecting the load across the electrode gap by measurement of a current signal.

17. The method of claim 15 wherein said biological cells comprise mammalian progenitor cells and/or stem cells.
